(19) United States
(12) Patent Application Publication
(10) Pub. No.: US 2011/0197887 A1
(43) Pub. Date: Aug. 18, 2011

(54) ACCESSORY CONNECTION AND DATA SYNCHRONICATION IN A VENTILATOR

(75) Inventors: William A. Truschel, Oakmont, PA (US); Andrew L. Shissler, Delmont, PA (US); Rochelle Siirola, Albuquerque, NM (US); Mark C. Mcdermott, Pittsburgh, PA (US); Michael H. Kissel, Harrison City, PA (US)

(73) Assignee: KONINKLIJKE PHILIPS ELECTRONICS N.V., EINDHOVEN (NL)

(21) Appl. No.: 13/123,961

(22) PCT Filed: Oct. 9, 2009
(86) PCT No.: PCT/IB2009/054450

§ 371 Date: Apr. 13, 2011

Related U.S. Application Data
(60) Provisional application No. 61/105,883, filed on Oct. 16, 2008.

(51) Int. Cl. A61M 16/00

(52) U.S. Cl. 128/204.23; 128/204.21

(57) ABSTRACT

A medical ventilator that includes a memory device, a controller operatively coupled to the memory device, and an accessory connector provided at or about the exterior of the ventilator housing. The controller is adapted to record ventilation data in the memory device that includes at least one of data relating to the operation of the ventilator and data relating to the breathing of the patient while ventilation therapy is being provided to the patient. The accessory connector is structured to operatively couple the controller to an accessory device and to provide power to the accessory device. The accessory connector also serves as a communication bus for enabling the accessory device to communicate data generated by it to the controller, which in turn is adapted to record the accessory device data in the memory device in a manner wherein the accessory device data is merged.

This patent application claims the priority benefit under 35 U.S.C. §119(e) of U.S. Provisional Application No. 61/105,883 filed on Oct. 16, 2008, the contents of which are herein incorporated by reference.

The present invention relates to a medical ventilator, and in particular to a medical ventilator that includes an accessory connector that enables data to be received from an accessory device operatively coupled to the accessory connector and integrated into a data stream being collected and/or generated by the medical ventilator.

A medical ventilator is a machine that is structured to deliver a gas, such as air, oxygen, or a combination thereof, to an airway of patient to augment or substitute for the patient's own respiratory effort. In addition, it is known to operate a conventional medical ventilator in a variety of modes depending upon the particular needs of the patient.

In a life support situation, where there is substantially no spontaneous respiratory effort by the patient, a controlled mode of ventilation is typically provided, where the ventilator assumes full responsibility for ventilating the patient. In this mode of ventilation, a controlled volume of gas is delivered to the patient during each inspiratory phase of the ventilatory cycle, and the trigger point (the transition from the expiratory phase to the inspiratory phase of the ventilatory cycle) and cycle point (the transition from the inspiratory phase to the expiratory phase of the ventilatory cycle) of the ventilator are typically determined based on time. Traditionally, ventilators used in life support situations employ what is known as a dual-limb patient circuit having an inspiratory limb for carrying gas to the patient and an expiratory limb for carrying gas from the patient to an exhaust assembly that includes a selectively controllable valve or similar mechanism for actively controlling the exhaustion of the patient's expired gas to atmosphere (referred to as “active exhaust”).

In non-life support situations, where the patient exhibits some degree of spontaneous respiratory effort, an assist mode or a support mode of ventilation is typically provided in which the ventilator augments or assists in the patient's own respiratory efforts, typically by providing a predetermined pressure to the airway of the patient. Ventilators used in non-life support situations typically employ what is known as a single-limb patient circuit having only one limb that is used for transporting gas both to and from the patient. In addition, such single-limb patient circuits normally include an exhaust port, often in the form of a hole in the limb, to allow the patient's expired gas to be passively vented to atmosphere (referred to as “passive exhaust”).

During operation, current ventilators (both those used in life support situations and those used in non-life support situations) record various types of data, such as certain waveform data relating to the ventilation therapy being provided to the patient and/or certain other detailed operational and event data, so that such data can be viewed, examined and evaluated at a later time by a caregiver. The data storage and data management capabilities of current ventilators, however, are somewhat limited.

Accordingly, it is an object of the present invention to provide a ventilator that overcomes the shortcomings of conventional ventilator. This object is achieved according to one embodiment of the present invention by providing a ventilator that includes (a) a housing having an interior and an exterior; (b) an inlet port extending from the exterior to the interior of the housing; (c) a flow generator, such as, without limitation, a blower, disposed within the housing and that is structured to generate a flow of gas; (d) an outlet port adapted to discharge the flow of gas from the housing; (e) a patient circuit, such as a single-limb or dual-limb patient circuit, in fluid communication with the outlet port that is structured to deliver the flow of gas to an airway of a patient during an inspiratory phase of the ventilatory cycle; (f) a memory device, such as, without limitation, a removable memory device like an SD card or similar device; and (g) a controller operatively coupled to the memory device. The ventilator also includes (h) an accessory connector provided at or about the exterior of the housing.

The controller is adapted to record ventilation data in the memory device that includes at least one of data relating to the operation of the ventilator and data relating to the breathing of the patient while ventilation therapy is being provided to the patient. In addition, the accessory connector is structured to operatively couple the controller to an accessory device, such as, without limitation, an accessory medical device such as a pulse oximeter to a carbon dioxide monitor. The accessory connector is further structured to provide power from the ventilator to the accessory device for powering the accessory device. Furthermore, the accessory connector and serves as a communication bus for enabling the accessory device to communicate accessory device data to the controller. The accessory device data is data this is generated by the accessory device while ventilation therapy is being provided to the patient. The controller is adapted to record the accessory device data in the memory device in a manner wherein the accessory device data is merged with the ventilation data such that the accessory device data is time-synchronized with the ventilation data.

It can thus be appreciated that the present invention enables one or more accessory devices used during patient treatment, such as a pulse oximeter, to be operatively coupled to the ventilator in a manner wherein the data is collected and received from the one or more accessory devices and that data is integrated into the data stream recorded by the ventilator.

The communication bus provided by the accessory connector may be a serial communication bus, such as, without limitation, a serial communication bus that is structured to provide multidrop communications according to a multidrop communications protocol, such as the RS-485 protocol, so that at least one additional accessory device may be operatively coupled to the controller for enabling the at least one additional accessory device to communicate data to the controller. In such as case, power is provided from the ventilator to the at least one additional accessory device through the accessory connector.

In response to the accessory device data being merged with the ventilation data, an output, including a representation of the ventilation data and a representation of the accessory device data, may be generated, wherein the waveform data is time-synchronized with the accessory device data. The ventilation data may be waveform data useable to generate a waveform relating to the operation of the ventilator or the breathing of the patient. Such waveform data may include data selected from the group consisting of (1) patient pressure data, (2) exhaled tidal volume data, (3) uncompensated flow data, (4) leak data, and (5) patient breath rate data.

In another embodiment, the present invention provides a method of operating a ventilator that includes steps of (a) operatively coupling an accessory device to an accessory connector located at or about an exterior of a ventilator, wherein the accessory connector serves as a communication bus to enable the accessory device to communicate with the ventilator; (b) providing power from the ventilator to the accessory device through the accessory connector; (c) providing ventilation therapy to a patient through the ventilator; (d) recording ventilation data, as described above, in a memory device of the ventilator, (e) receiving accessory device data, as described above, in the ventilator through the accessory connector; and (f) recording the accessory device data in the memory device in a manner wherein the accessory device data is merged with the ventilation data such that the accessory device data is time-synchronized with the ventilation data.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

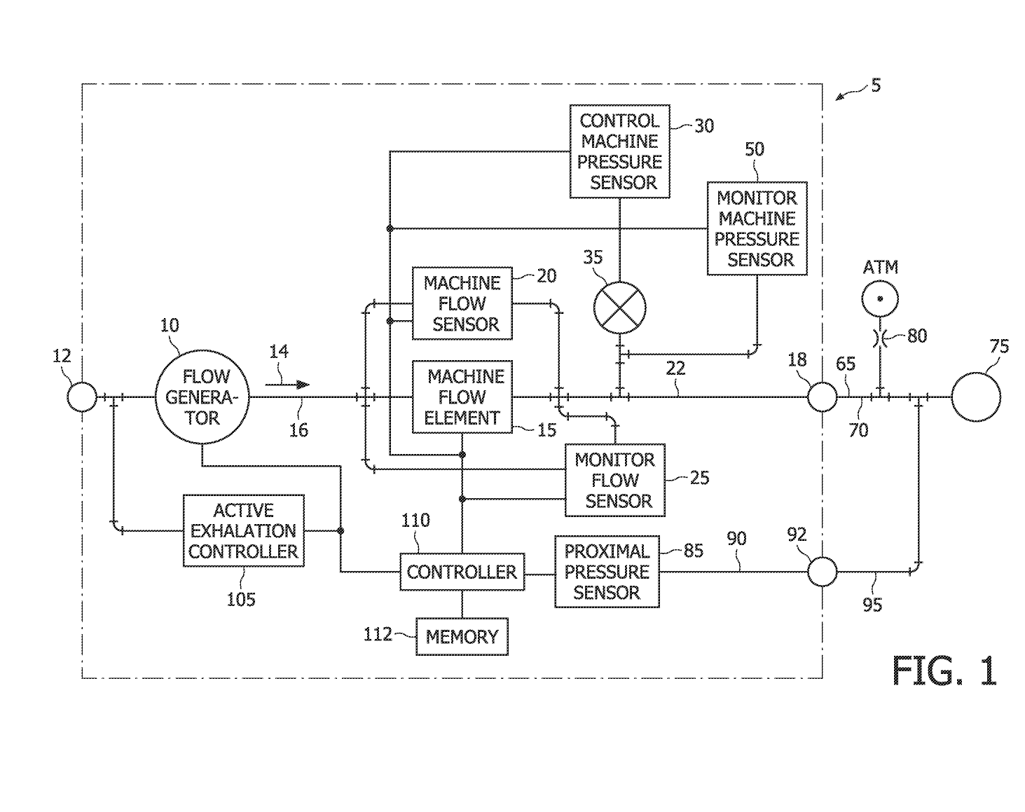

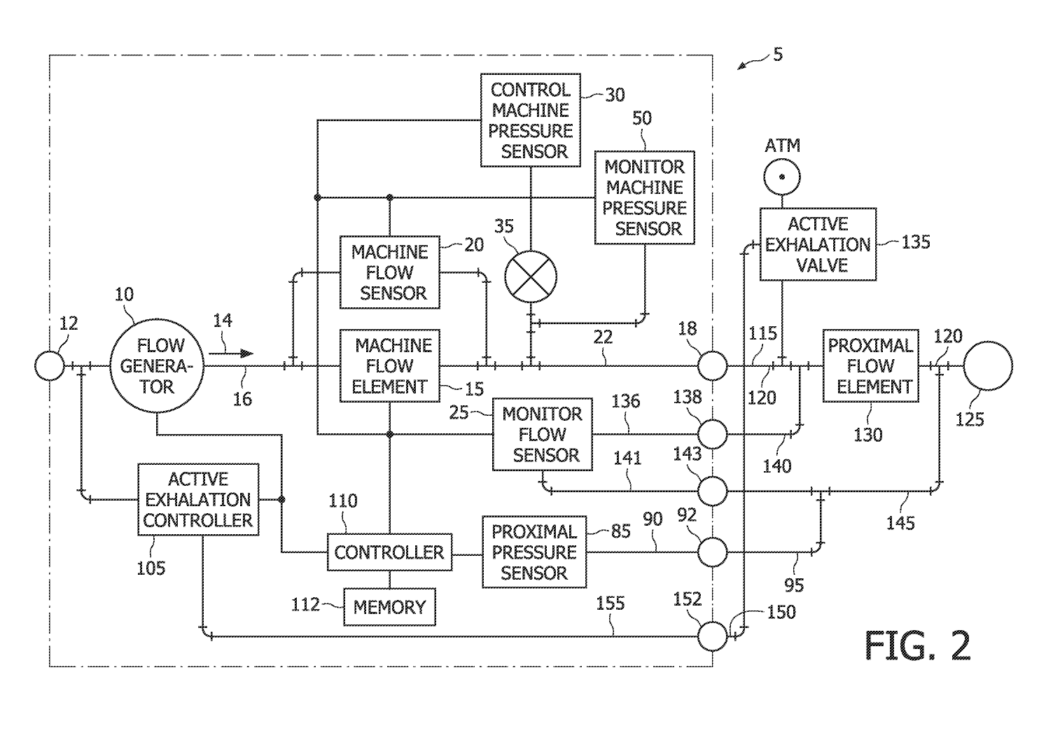

FIGS. 1 and 2 are schematic diagrams of an illustrative embodiment of a ventilator in which the present invention may be implemented;

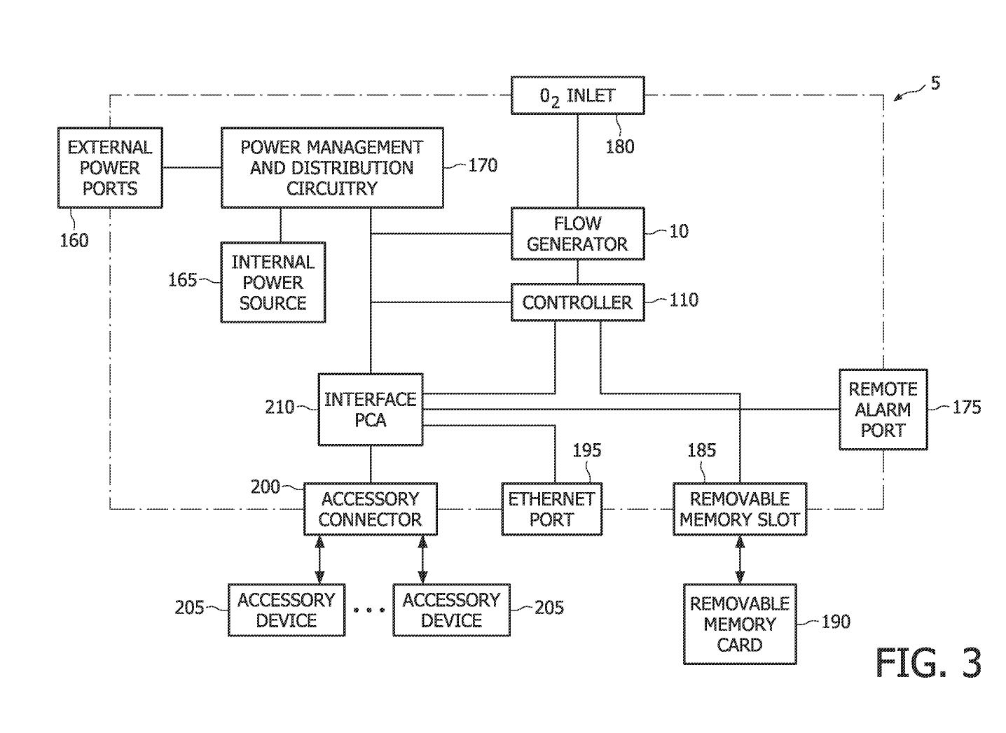

FIG. 3 is a schematic diagram showing selected external interfaces, including the accessory connector of the present invention, and selected internal components included as part of the ventilator embodiment shown in FIGS. 1 and 2;

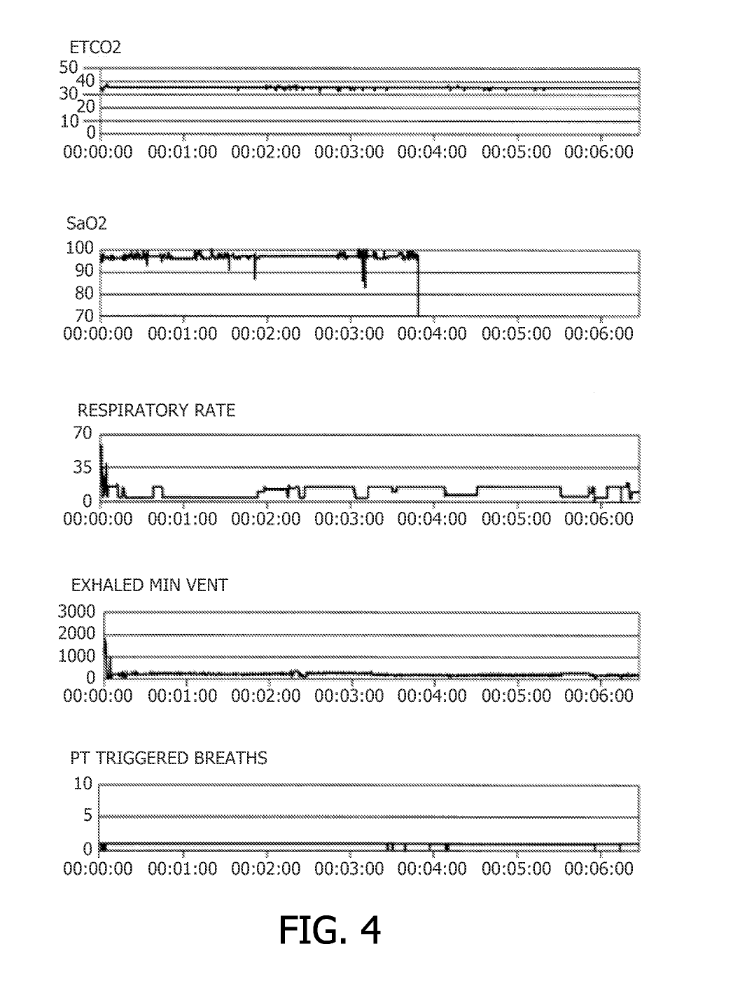

FIG. 4 shows an exemplary output generated from the data saved in the memory of the ventilator shown in FIGS. 1, 2, and 3, which includes waveforms generated from both waveform data generated by the ventilator and data received from an accessory device operatively coupled to the accessory connector of the ventilator embodiment shown in FIGS. 1, 2, and 3.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed herein, the term “patient interface” refers to any known or suitable mechanism for transporting gas to and from the airway of a patient and expressly includes, but is not limited to, non-invasive patient interfaces such as masks, nasal canulas, combination nasal/oral masks and removable mouth pieces, and invasive patient interfaces such as tracheal tubes and endotracheal tubes, as well as humidifiers, nebulizers and meter dose inhalers, which can be invasive or non-invasive.

As employed herein, the term “mode” refers to the operation of the ventilator for providing a particular type of ventilation therapy, expressly including but not limited to, pressure support ventilation therapy, volume control ventilation therapy and suitable combinations thereof. Each mode may have one or more attributes such as, for example and without limitation, CPAP, SIMV, S, S/T, AC, PC, PC-SIMV, or CV.

As employed herein, the statement that two or more parts or components are “coupled” together shall mean that the parts are joined or operate together either directly or through one or more intermediate parts or components.

As employed herein, the term “number” shall mean one or an integer greater than one (i.e., a plurality).

The present invention provides a medical ventilator that includes an external interface in the form of an accessory connector that enables data collected and received from an accessory device or devices operatively coupled to the accessory connector to be integrated into the data stream being generated by the medical ventilator. FIGS. 1 and 2 are schematic diagrams of an illustrative embodiment of a particular ventilator 5 in which the present invention may be implemented.

As described in greater detail below, ventilator 5 in FIGS. 1 and 2 is capable of being selectively configured to provide ventilation to a patient in a number of different modes, including volume controlled and pressure support modes, using either passive or active exhaust and a single-limb patient circuit. It should be understood, however, that ventilator 5 shown in FIGS. 1 and 2 and described in greater detail below is being used for illustrative purposes only in order to describe an implementation of the present invention, and that the invention as described herein and defined by the claims hereof may be implemented in other types of ventilators having various other capabilities and modes of operation. Ventilator 5 should therefore not be considered to be limiting.

In FIG. 1, ventilator 5 is shown in a configuration in which passive exhaust is employed. Ventilator 5 includes within a housing a flow generator 10 adapted to generate a flow of gas, such as air from an ambient air inlet port 12 (extending from the exterior to the interior of the housing) and/or a mixture of air and oxygen provided from ambient air inlet port 12 and an optional oxygen source (not shown). Flow generator 10 may be any device suitable for creating a flow of gas (indicated by the arrow 14) at a pressure greater than ambient atmosphere, such as a compressor, fan, impeller, blower, piston or bellows. In the preferred embodiment, flow generator 10 is a micro-turbine comprising a blower assembly having a brushless DC motor with an impeller design to generate the pressures and flows required by the ventilator 5. Flow generator 10 is in fluid communication with a machine flow element 15 through a conduit 16. Machine flow element 15 is a mechanical element positioned at or about the outlet of the flow generator that is designed to produce a pressure drop when flow passes through it. As seen in FIG. 1, machine flow element 15 is in fluid communication with an outlet port 18 of ventilator 5 through a conduit 22.

A machine flow sensor 20 is provided in tandem with machine flow element 15 to measure volumetric flow of the flow of gas created by the flow generator 10. In addition, a monitor flow sensor 25 is also provided in tandem with machine flow element 15 to monitor the machine volumetric flow in a redundant manner. Preferably, one or both of machine flow sensor 20 and monitor flow sensor 25 is a differential pressure sensor. Furthermore, machine flow sensor 25 may be used in tandem with a proximal pressure sensor 85 (FIG. 2) to measure volumetric flow from the patient during exhalation and to provide improved triggering sensitivity and accuracy of the exhaled tidal volume. It can be appreciated that the ventilator need not have both flow sensors. In addition, the present invention even further contemplates eliminating both flow sensors in favor of measuring the flow rate, or a parameter indicative of the flow rate, using other techniques, such as based on the power provided to flow generator, the speed of the flow generator, etc.

A control machine pressure sensor 30 is operatively coupled to the conduit 22 through an auto zero valve 35. Control machine pressure sensor 30 is preferably a static pressure sensor and is used to monitor the pressure at the outlet port 18 of the ventilator 5. In addition, a monitor machine pressure sensor 50 is operatively coupled to the conduit 22 and is also preferably a static pressure sensor used to monitor the pressure at the outlet port 18 of the ventilator 5 in a redundant fashion. It can be appreciated that the ventilator need not have both pressure sensors.

As seen in FIG. 1, a single-limb patient circuit 65 is in fluid communication with outlet port 18 of ventilator 5 and includes a conduit 70 and a patient connection port 75 adapted to the connected to a patient interface assembly, such as a mask, mouthpiece, combination nasal/oral mask, full face mask, tracheal tube, or endotracheal tube, for delivering the flow of gas to the airway of the patient. Single-limb patient circuit 65 in the embodiment shown in FIG. 1 includes a passive exhalation valve 80 for venting gas expired by the patient to the atmosphere. Furthermore, ventilator 5 in this embodiment includes a proximal pressure sensor 85 that is in fluid communication with the single-limb patient circuit 65 through an internal conduit 90, a port 92, and an external conduit 95. In an exemplary embodiment, proximal pressure sensor 85 is a static pressure sensor used to measure delivered gas pressure at the patient connection port 75.

Although not employed in the configuration of ventilator 5 shown in FIG. 1, the ventilator includes an active exhalation controller 105 (described in more detail below) that is used when the ventilator is configured as shown in FIG. 2 to provide for active exhaust. Finally, the ventilator shown in FIG. 1 includes a controller 110, such as a microprocessor, a microcontroller or some other suitable processing device, that is operatively coupled to a memory 112. Memory 112 can be any of a variety of types of internal and/or external storage media, such as, without limitation, RAM, ROM, EPROM(s), EEPROM(s), and the like, that provide a storage medium for data and software executable by controller 110 for controlling the operation of the ventilator as described herein. As shown in FIG. 1, processor 110 is in electronic communication with certain of the other components shown in FIG. 1 in order to control such components and/or receive data from such components. For example, data may be transmitted from the various sensors of the ventilator 5 to the controller 110 so that such data may be manipulated and/or logged as described elsewhere herein (FIG. 3).

Referring to FIG. 2, ventilator 5 is shown in a configuration adapted for providing active exhaust. Thus, as seen in FIG. 2, ventilator 5 includes an alternate single-limb patient circuit 115 in fluid communication with outlet port 18. This single-limb patient circuit 115 includes a conduit 120, a patient connection port 125 similar to the patient connection port 75, a proximal flow element 130, and an active exhalation valve 135. Proximal flow element 130 is a mechanical element positioned at or about patient connection port 125 that is designed to produce a pressure drop when flow passes through it. Active exhalation valve 135 is preferably a proportionally controlled pressure relief valve in single-limb patient circuit 115 that provides for low resistance and includes carbon dioxide flushing during patient exhalation. In addition, active exhalation valve 135 preferably provides for low exhalation resistance in the event of loss of therapy to meet anti-asphyxia requirements.

As seen in FIG. 2, in the configuration shown therein, monitor flow sensor 25 is operatively coupled to conduit 120 at either end of proximal flow element 130 rather than being operatively coupled to the conduits 16 and 22 as in the configuration of FIG. 1. In particular, monitor flow sensor 25 is operatively coupled at a first end of proximal flow element 130 through an internal conduit 136, a port 138, and an external conduit 140, and at a second end of the proximal flow element through an internal conduit 141, a port 143, and an external conduit 145. Furthermore, active exhalation controller 105 in this configuration is operatively coupled to the active exhalation valve 135 by way of an external conduit 150, a port 152 and/or internal conduit 155.

Active exhalation controller 105 is preferably a pressure control unit that regulates the pilot pressure of active exhalation valve 135 diaphragm in order to control bias flow during patient exhalation. Active exhalation controller 135 preferably includes a dump valve to quickly reduce the pilot pressure from the diaphragm of the active exhalation valve to allow it to fully open at the beginning of exhalation. Active exhalation controller 105 also preferably includes a proportional valve that is used in combination with an orifice provided between the two valves to control bias flow.

In still a further embodiment, the configuration of the ventilator shown in FIG. 1 may be altered so as to not include the operative coupling between proximal pressure sensor 85 and single limb circuit 65. As will be appreciated, such a configuration would not include control based on measured proximal pressure.

Again, as noted above, ventilator 5 shown in FIGS. 1 and 2 as just described is being used herein for illustrative purposes only in order to describe an embodiment employing the present invention, and it should be understood that the invention described herein may be employed in other ventilator types and/or configurations and is not intended to be limited to use with the ventilator.

Although not shown, the present invention contemplates that ventilator 5 includes an input/output component (e.g., user interface) or components. The input/output component is used, for example, for setting various parameters used by the ventilator as well as for displaying and outputting information and data to a user. The input/output component may be any device suitable to provide information and/or commands to controller 110 via an operative link and to present information to the patient, or another user, in a human perceivable format. Examples of a suitable input/output device includes a keypad, keyboard, touch pad, mouse, visual display (e.g., LCD or LED screen), microphone, speaker, switches, button, dials, lamps, or any other devices that allow a user to input information to and receive information from the ventilation system. The present invention further contemplates providing a wireless link as an input/output component to enable remote communication with the ventilator wirelessly.

FIG. 3 is a schematic diagram showing selected external interfaces included as part of ventilator 5 along with selected internal components of the ventilator. As seen in FIG. 3, ventilator 5 includes external power ports 160 for connecting the ventilator to one or more sources of power, such as, for example, an AC power source, an external lead-acid battery, and/or an external rechargeable (e.g., Li-ion) battery. The ventilator also includes an internal power source 165, such as an internal rechargeable (e.g., Li-ion) battery. External power ports 160 and internal power source 165 are operatively coupled to power management and distribution circuitry 170 which, among other things, manages the use of the power supplies just described and distributes a regulated voltage of an appropriate level to power the various components of the ventilator. The ventilator also includes a remote alarm port 175 which provides an interface for connection to a nurse call or similar remote alarm system. An oxygen inlet 180 is provided to enable oxygen to be added to the flow of gas generated by flow generator 10.

As also seen in FIG. 3, ventilator 5 includes a removable memory slot 185 structured to receive therein a removable memory card 190, such as an SD (secure digital) card or similar memory device. Removable memory card 190 is provided to store certain log data (for clinical and diagnostic purposes) generated by the ventilator during the operation thereof and may also be used to provide prescription information (new or updated) and/or new program software for the ventilator (which may be, for example, downloaded through the Internet through the Ethernet port 195 described below). In particular, the ventilator is adapted to record waveform data which may be used to generate one or more waveforms to the removable memory card 190 while the flow generator 10 is turned on. Such waveform data may include, without limitation, patient pressure data recorded periodically, such as every 100 ms, exhaled tidal volume data recorded periodically, such as every 100 ms, uncompensated flow data recorded periodically, such as every 100 ms, ventilator leak data recorded periodically, such as every 100 ms, and patient breath rate data recorded periodically, such as every second, among others.

In an exemplary embodiment of the prevent invention, ventilator 5 is adapted to record a minimum amount of waveform data, such as 72 hours worth of data, to removable memory card 190 during operation of the ventilator. In addition, ventilator 5 is also adapted to record certain detailed data relating to the operation of the ventilator to removable memory card 190 while flow generator 10 is operating. For example, such detailed data may include, without limitation, average (over some predetermined interval such as 30 seconds) attained pressures (e.g., IPAP (inspiratory positive airway pressure), EPAP (expiratory positive airway pressure), CPAP (continuous positive airway pressure), PEEP (positive end expiratory pressure) as appropriate for therapy mode), average breath rate, average [percentage of patient triggered breaths, average peak inspiratory patient flow, average total leak, average exhaled tidal volume, and average exhaled minute ventilation, among others.

In a further exemplary embodiment, the ventilator is adapted to record a minimum amount, such as one years worth, of such detailed data to removable memory card 190. Furthermore, the ventilator is adapted to record certain annotation data to removable memory card 190 during operation thereof. Such annotation data may include, for example, and without limitation, flow generator on and off events, current prescription settings, prescription setting changes, patient alarm settings, and patient alarm occurrences, among others. Preferably, the ventilator is adapted to record the annotation data to correspond with the recorded detailed data.

As seen in FIG. 3, removable memory slot 185 is in electronic communication with controller 110 to enable the controller to selectively record data to the removable memory card. In an alternative embodiment, a non-removable memory device internal to the ventilator may be employed instead of removable memory card 190.

Furthermore, ventilator 5 includes an Ethernet port 195 that is in electronic communication with controller 110 to enable the ventilator to make a high speed direct connection to an Ethernet network. As will be appreciated, Ethernet port 195 thus enables the ventilator to communicate with devices, such as remotely located devices, which are connected to the Ethernet network.

As seen in FIG. 3 and as described elsewhere herein, ventilator 5 includes a sensor printed circuit assembly (PCA) which includes the various sensors described elsewhere herein and which operatively couples those sensors to controller 110. This connection provides much of the data that is ultimately stored on removable memory card 190.

Finally, ventilator 5 includes an accessory connector 200 which both serves as a serial communication bus to enable one or more accessory devices 205, such as, without limitation, a pulse oximeter or a carbon dioxide monitor, to the ventilator and provides a regulated power output, preferably a 24 volt regulated and current limited output, to power accessory devices 205. In the preferred embodiment, accessory connector 200 provides an RS-232/RS-485 serial interface for the ventilator, which is designed for multi-drop communications so that more than one accessory device 205 can share the same connection.

An interface printed circuit assembly (PCA) 210 is provided and is in electronic communication with controller 110, remote alarm port 175, Ethernet port 195, and accessory connector 200 for operatively coupling those components to the controller. In addition, interface PCA 210 provides the regulated voltage output described above to accessory connector 200, and supports RS-232 communications, RS-485 communications, Ethernet communications, and the interface to remote alarm port 175. Interface PCA 210 may also include an oxygen sensor (not shown) for oxygen leak detection.

According to an aspect of the present invention, data is collected from one or more accessory devices 205 through accessory connector 200 and is recorded on removable memory card 190. In particular, the data from accessory devices 205 is recorded in a manner where it is seamlessly and automatically merged with the data described elsewhere herein (the waveform data, the detailed data and/or the annotation data) that is recorded on the removable memory card in a time synchronous manner. As a result, the waveforms and other outputs for data analysis and reporting that are provided from or derived from the data on removable memory card 190 may be selectively accessed based on not only the data that is generated by the by the ventilator relating to the operation thereof (the waveform data, the detailed data and/or the annotation data), but also on the data that is collected from the one or more accessory devices 205. In operation, the software running on controller 110 will automatically detect when an accessory device 205 is operatively coupled to accessory connector 200 and will record the data therefrom to removable memory card 190 as described above. In addition, when an accessory device 205 is removed from accessory connector 200, the software will automatically remove that channel from the data configuration.

For example, in one particular embodiment, accessory devices 205 include both a pulse oximeter and a carbon dioxide monitor. As described above, the oxygen saturation data from the pulse oximeter (SaO₂) and the end tidal carbon dioxide data (ETCO₂) from the carbon dioxide monitor will be recorded on removable memory card 190 and will be merged with the data that is generated by the by ventilator 5 relating to the operation thereof (the waveform data, the detailed data and/or the annotation data). As a result, an output that includes waveforms for, for example, patient respiratory rate, patient exhaled minute ventilation, and percentage of patient triggered breaths time synchronized with waveforms for SaO₂ and ETCO₂ such as is shown in FIG. 4 may be generated from the data on the removable memory card 190. As will be appreciated, the merged data and outputs generated therefrom is extremely helpful to a caregiver in treating the patient. It should be understood, however, that the output shown in FIG. 3 is meant to be exemplary only, and that outputs including numerous other combinations of merged data may also be selectively generated according to the present invention.

Moreover, it is to be understood that the schematic diagrams of ventilator 5 shown in FIGS. 1, 2, and 3 are not intended to be a complete and exhaustive description of the ventilator 5, but instead are intended to describe the key components of the ventilator, especially those necessary to implement and carry out the present invention. Those skilled in the art would understand, for example, that a medical ventilator system could also include features such as an input/output device for setting the operating parameters of the system, alarms (audible or visual) for signaling conditions of the patient or ventilator to an operator, as well as ancillary elements connected to the patient circuit, such as a humidifier, bacteria filter, an aspiration catheter, and a tracheal gas insufflation catheter, to name a few.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A ventilator comprising:
(a) a housing having an interior and an exterior;
(b) an inlet port extending from the exterior to the interior of the housing;
(c) a flow generator disposed within the housing and being structured to generate a flow of gas;
(d) an outlet port adapted to discharge the flow of gas from the housing;
(e) a patient circuit in fluid communication with the outlet port and being structured to deliver the flow of gas to an airway of a patient during an inspiratory phase of a ventilatory cycle;
(f) a memory device;
(g) a controller operatively coupled to the memory device and being adapted to record ventilation data in the memory device, the ventilation data comprising at least one of data relating to the operation of the ventilator and data relating to the breathing of the patient while ventilation therapy is being provided to the patient; and
(h) an accessory connector provided at or about the exterior of the housing, the accessory connector being structured to operatively couple the controller to an accessory device, the accessory connector being further structured to both provide power from the ventilator to the accessory device for powering the accessory device and serving as a communication bus for enabling the accessory device to communicate accessory device data to the controller, the accessory device data being generated by the accessory device while ventilation therapy is being provided to the patient, wherein the controller is adapted to record the accessory device data in the memory device in a manner wherein the accessory device data is merged with the ventilation data such that the accessory device data is time-synchronized with the ventilation data.

2. The ventilator according to claim 1, wherein the communication bus is a serial communication bus.

3. The ventilator according to claim 2, wherein the serial communication bus is structured to provide multidrop communications according to a multidrop communications protocol so that at least one additional accessory device may be operatively coupled to the controller for enabling the at least one additional accessory device to communicate data to the controller, wherein power is provided from the ventilator to the at least one additional accessory device through the accessory connector.

4. The ventilator according to claim 3, wherein the multidrop communications protocol is the RS-485 protocol.

5. The ventilator according to claim 1, wherein in response to the accessory device data being merged with the ventilation data, an output including a representation of the ventilation data and a representation of the accessory device data may be generated wherein the waveform data is time-synchronized with the accessory device data.

6. The ventilator according to claim 1, wherein the ventilation data is waveform data useable to generate a waveform relating to the operation of the ventilator or the breathing of the patient, and wherein, in response to the accessory device data being merged with the ventilation data, an output including the waveform and a representation of the accessory device data may be generated wherein the waveform data is time-synchronized with the accessory device data.

7. The ventilator according to claim 6, wherein the waveform data includes data selected from the group consisting of (1) patient pressure data, (2) exhaled tidal volume data, (3) uncompensated flow data, (4) leak data, and (5) patient breath rate data.

8. The ventilator according to claim 1, further comprising a memory device slot accessible from the exterior of the housing, wherein the memory device is a removable memory device inserted within the memory device slot.

9. The ventilator according to claim 8, wherein the memory device slot is an SD card slot and wherein the removable memory device is an SD card.

10. The ventilator according to claim 1, wherein the accessory device is a medical device.

11. The ventilator according to claim 10, wherein the medical device is a pulse oximeter or a carbon dioxide monitor.

12. A method of operating a ventilator, comprising:
(a) operatively coupling an accessory device to an accessory connector located at or about an exterior of a ventilator, the accessory connector serving as a communication bus to enable the accessory device to communicate with the ventilator;
(b) providing power from the ventilator to the accessory device through the accessory connector;
(c) providing ventilation therapy to a patient through the ventilator;
(d) recording ventilation data in a memory device of the ventilator, the ventilation data comprising at least one of data relating to the operation of the ventilator and data relating to the breathing of the patient while ventilation therapy is being provided to the patient;
(e) receiving accessory device data in the ventilator through the accessory connector, the accessory device data being generated by the accessory device while the ventilation therapy is being provided to the patient; and
(f) recording the accessory device data in the memory device in a manner wherein the accessory device data is merged with the ventilation data such that the accessory device data is time-synchronized with the ventilation data.

13. The method according to claim 12, wherein the communication bus is a serial communication bus.

14. The method according to claim 13, wherein the serial communication bus is structured to provide multidrop communications according to a multidrop communications protocol, wherein the method further comprises operatively coupling at least one additional accessory device to the accessory connector for enabling the at least one additional accessory device to communicate data to the ventilator.

15. The method according to claim 14, further comprising providing power to the at least one additional accessory device through the accessory connector.

16. The method according to claim 14, wherein the multidrop communications protocol is the RS-485 protocol.

17. The method according to claim 12, wherein in response to the accessory device data being merged with the ventilation data, an output including a representation of the ventilation data and a representation of the accessory device data may be generated wherein the waveform data is time-synchronized with the accessory device data.

18. The method according to claim 17, further comprising generating the output.

19. The method according to claim 12, wherein the ventilation data is waveform data useable to generate a waveform relating to the operation of the ventilator or the breathing of the patient, and wherein, in response to the accessory device data being integrated with the ventilation data, an output including the waveform and a representation of the accessory device data may be generated wherein the waveform data is time-synchronized with the accessory device data.

20. The method according to claim 19, wherein the waveform data includes data selected from the group consisting of (1) patient pressure data, (2) exhaled tidal volume data, (3) uncompensated flow data, (4) leak data, and (5) patient breath rate data.

21. The method according to claim 20, wherein the memory device is a removable memory device, the method further comprising removing the memory device from the ventilator and generating the output from the waveform data and the accessory device data.

22. The method according to claim 21, wherein the removable memory device is an SD card.

23. The method according to claim 12, wherein the accessory device is a medical device selected from the group consisting of a pulse oximeter and a carbon dioxide monitor.
